(19) United States
(12) Patent Application Publication
(10) Pub. No.: US 2024/0001049 A1
(43) Pub. Date: Jan. 4, 2024

(54) Smart Metered Pulmonary Delivery Device System

(71) Applicant: Arthur Jaffee, Denver, CO (US)

(72) Inventor: Arthur Jaffee, Denver, CO (US)

(21) Appl. No.: 18/218,002

(22) Filed: Jul. 3, 2023

Related U.S. Application Data
(60) Provisional application No. 63/358,148, filed on Jul. 3, 2022.

(51) Int. Cl. A61M 11/00; A24F 40/10; A24F 40/05; A24F 40/42; A24F 40/51; A24F 40/65; A24F 40/53; A24F 40/60

(52) U.S. Cl. CPC A61M 11/006; A24F 40/10; A24F 40/05; A24F 40/42; A24F 40/51; A24F 40/65; A24F 40/53; A24F 40/60; A61M 2205/3334; A61M 2205/50; A61M 2205/13; A61M 2205/609; A61M 2205/587; A61M 2205/581; A61M 2205/582; A61M 2205/3561

(57) ABSTRACT

A smart vaporizer that uses a vibrating mesh nebulizer with water based liquids that tracks the type of liquid, dosage inhaled, time, and user then transmits that information to smart devices such as smart phones and smart watches which then transmits back to the vaporizer additional data related to user weight, body fat, heart rate, blood pressure, temperature, movement, sleep, blood sugar, and medication usage to determine when metered amounts of said liquid may be inhaled by a user.

### BACKGROUND OF THE INVENTION

### 1. Field of the Invention

The present invention is in the technical field of vapor inhalers. More particularly, the present invention pertains to the field of handheld heatless metered micro-nebulizing handheld water vaporization device with precise metered delivery of dosages of chemicals with connectivity to a health monitoring device with biometric and biomedical sensor remote patient monitoring feedback.

### 2. Description of Related Art

Vaporization devices provide effective delivery for a variety of chemicals and medications, such as vitamins, antibiotics, insulin, asthma medications, pain medications, erectile disfunction medications; CBD, THC, and nicotine. Such systems deliver the active ingredients directly to a user's respiratory system by vaporizing a desired dose of an active ingredient into a liquid form. A user then inhales the vaporized active ingredient(s) directly into the respiratory system, enabling said active ingredients to take effect faster.

Delivery of accurate and consistent metered doses of vaporized active ingredients to a user is very important. Current of vapor devices do not pair precise dosing administration information with biofeedback sensors in wearable devices such as heart sensors, temperature sensors, movement sensors, sleep sensors, blood sugar sensors, and tracking of medications and medical identifications such as those in current smart watches. The disclosure below solves this problem.

By pairing doses of aerosolized medication with health monitoring sensors, the user and medical providers are provided with multiple real-time data sets of biofeedback to understand the effect the chemicals are having on the user, allowing them to tailor the quantity and frequency of doses of aerosolized medication or chemicals to the individual. These and additional improvements to the art are described below.

### SUMMARY

The scope of the present invention is defined solely by the appended claims and detailed description of a preferred embodiment and is not affected to any degree by the statements within this summary. In addressing many of the problems experienced in the related art, such as those relating to obtaining biofeedback data in relation to administration of aerosolized chemicals and then applying that data to the administration of said chemicals, the present disclosure describes a vapor device system comprising one or more of the following elements: use of a high-frequency voltage-driven vibrating mesh nebulizer to turn a water-based mixture into vapor without the use of heat, use of a water-based mixture that can remain pressurized in contact with a nebulizer; precise control over the concentration of active ingredients in the liquid nebulized and the rate of vapor production allowing for dosage of active ingredients to be “metered”; production and administration of consistent ultra-fine liquid droplets, use of cartridge holding a fluid with a computer chip that communicates information to and from a main device housing electronically; a main device housing that with a computer chip that communicates information to and from secondary devices related to, but not limited to: dosage, frequency, heart rate, heart rhythm, temperature, blood sugar levels, movement; with visual, haptic, and audio indicators of dosage and frequency of dosage; with tracking of a user's identification, and prior use of medications, and programmable dosing and automatic stop features.

### BRIEF DESCRIPTION OF THE DRAWINGS

Various embodiments are described herein with reference to the following Drawings Not all alternatives and options are shown in the Drawings and, therefore, the Claims are not limited in scope to the content of the Drawings. Elements in the several figures are illustrated for simplicity and clarity and have not necessarily been drawn to scale. Also, common, but well-understood elements that are useful or necessary in commercially feasible embodiment are often not depicted to facilitate less obstructed views of these various embodiments of the present disclosure.

### 1. FIGURES

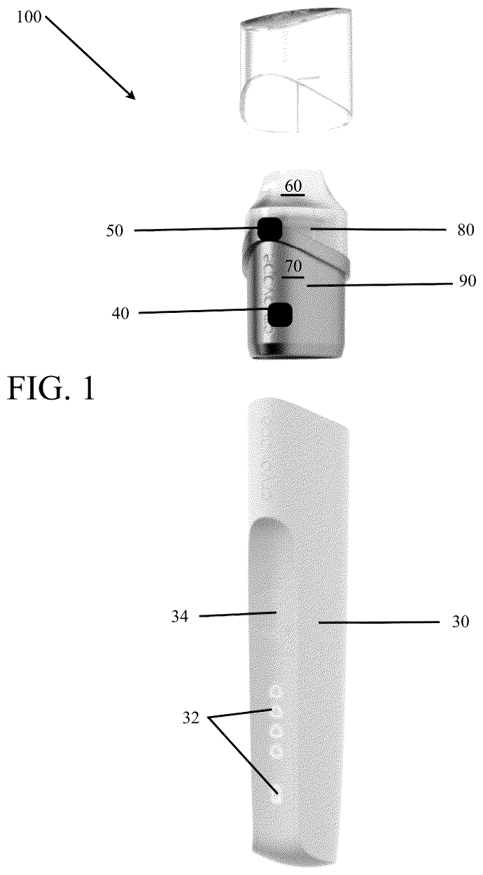

FIG. 1 illustrates a vapor device, in accordance with an embodiment of the present disclosure.

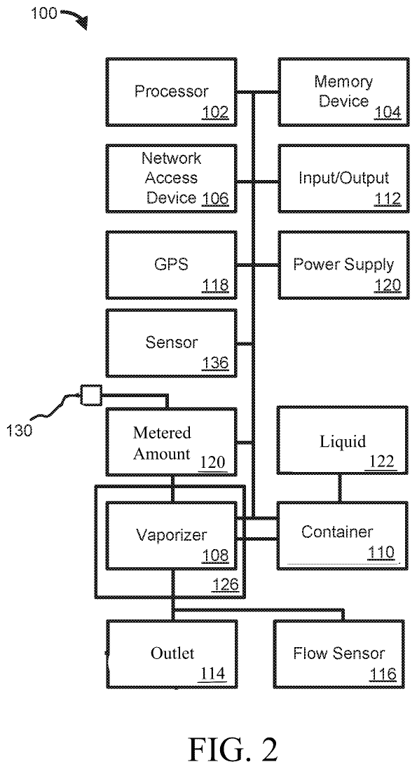

FIG. 2 illustrates a block diagram of vapor device system, in accordance with an embodiment of the present disclosure.

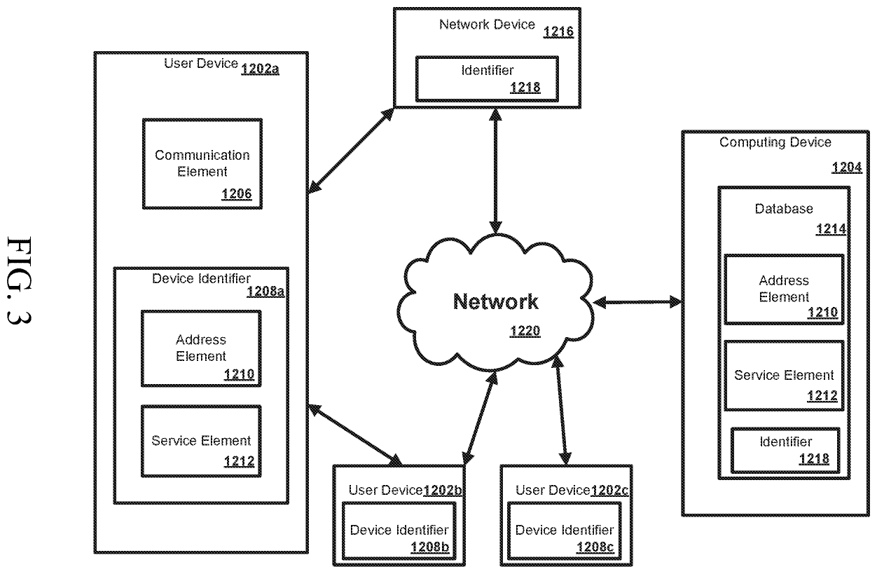

FIG. 3 illustrates an operating environment, in accordance with an embodiment of the present disclosure.

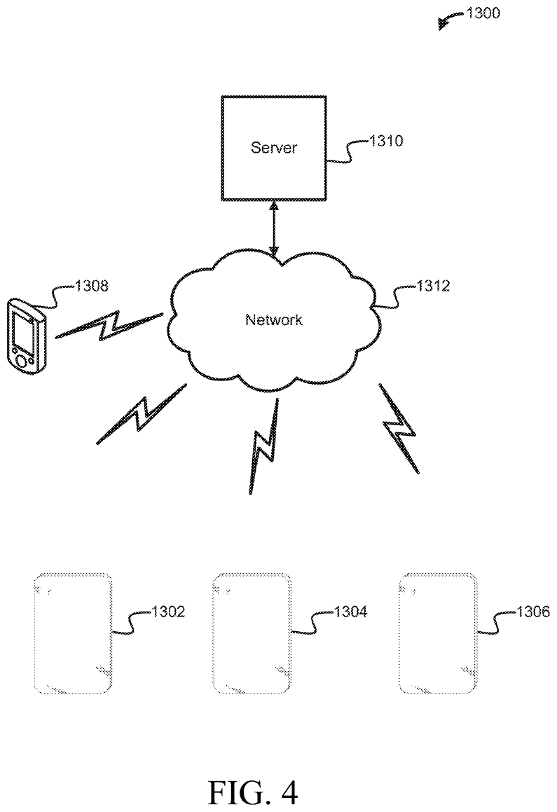

FIG. 4 illustrates an operating environment, in accordance with an embodiment of the present disclosure.

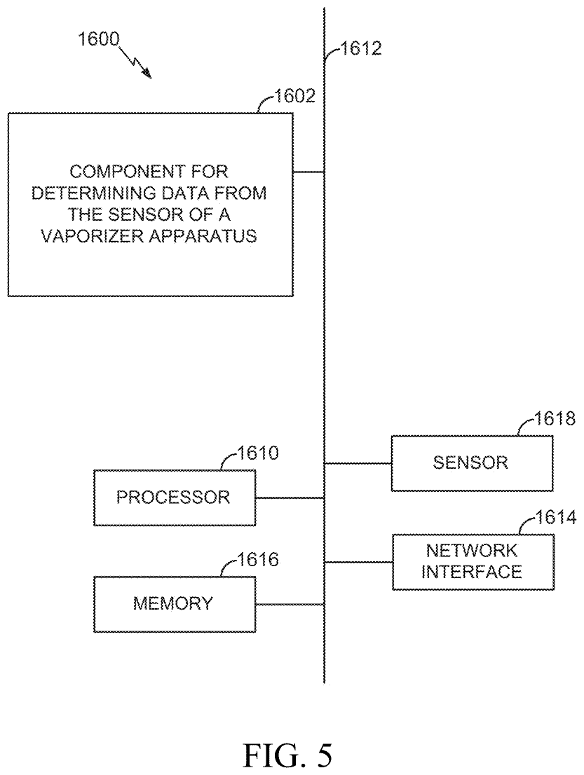

FIG. 5 illustrates a block diagram of operational features, in accordance with an embodiment of the present disclosure.

Corresponding reference characters indicate corresponding components throughout the several figures of the Drawings.

### DETAILED DESCRIPTION

The following description is not to be taken in a limiting sense but is made merely for the purpose of describing the general principles of exemplary embodiments, no limitation of the scope of the invention is thereby intended. The phrases: “in one embodiment,” “in an embodiment,” and similar language throughout this specification may, but do not necessarily, all refer to the same embodiment. The phrases “at least one,” “one or more,” and “and/or” are open-ended expressions that are both conjunctive and disjunctive in operation. The terms “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably and are understood to mean open sets of options; such as, the elements A+B and any additional element C. The described features, structures, methods, steps, or characteristics of the present disclosure may be combined in any suitable manner in one or more embodiments. In other instances, well-known structures, materials, or operations are not shown or described in detail to avoid obscuring aspects of the present disclosure. Further, all numbers expressing dimensions, physical characteristics, and so forth, used in the specification and claims are to be understood as being modified in all instances by the term “about”.

Regarding the illustrations, as used in the following description, the terms “horizontal”, “vertical”, “left”, “right”, “up” and “down”, as well as adjectival and adverbial derivatives thereof (e.g., “horizontally”, “rightward”, “upwardly”, etc.), simply refer to the orientation of the illustrated structure as the particular drawing or figure faces the reader. Similarly, the terms “inwardly” and “outwardly” generally refer to the orientation of a surface relative to its axis of elongation, or axis of rotation, as appropriate.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium can be utilized.

Embodiments of the methods and systems are described below with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions can be loaded onto a general-purpose computer, special purpose computer, or other programmable data processing apparatus to produce a machine, such that the instructions which execute on the computer or other programmable data processing apparatus create a feature for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of feature for performing the specified functions, combinations of steps for performing the specified functions and program instruction feature for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

For the purposes of promoting an understanding of the principles of the present invention, reference will now be made to the embodiments illustrated in the drawings and specific language will be used to describe the same.

FIG. 1 illustrates an embodiment of a fairly standard vapor device 100 comprising: a cartridge section 90 comprising: a reservoir 80 containing a liquid 70 to be vaporized and a mouthpiece 60 whereby a user may inhale vaporized liquid from the mouthpiece. The vapor device may also comprise a vibratable mesh 50, said vibratable mesh 50 being disposable between said reservoir and said mouthpiece, so as to vaporize liquid drawn from the reservoir to the mouthpiece. The vapor device may also comprise a flow sensor 40, wherein said flow sensor is arranged to detect a user drawing fluid through the mouthpiece during use and to actuate the vibrating mesh when fluid flow is detected. Additionally, there is a body section 30. On the body section there are one or more outputs 32, such as lights, sounds, haptics or led panels, and one or more inputs 24, such as buttons, touch screen, fingerprint detectors, or a microphone.

FIG. 2 illustrates a block diagram of another embodiment of a vapor device 100 and may comprise a processor 102. The processor 102 can be, or can comprise, any suitable microprocessor or microcontroller. The processor 102 can be coupled (e.g., communicatively, operatively, etc. . . . ) to auxiliary devices or modules of the vapor device 100 using a bus or other coupling. The vapor device 100 can comprise a power supply 120. The power supply 120 can comprise one or more batteries and/or other power storage device (e.g., capacitor) and/or a port for connecting to an external power supply. For example, an external power supply can supply power to the vapor device 100 and a battery can store at least a portion of the supplied power.

The vapor device 100 may comprise a memory device 104 coupled to the processor 102. The memory device 104 may comprise random-access memory (RAM) configured for storing program instructions and data for execution or processing by the processor 102 during control of the vapor device 100. When the vapor device 100 is powered off or in an inactive state, program instructions and data can be stored in a long-term memory. Either or both of the RAM or the long-term memory can comprise a non-transitory computer-readable medium storing program instructions that, when executed by the processor 102, cause the vapor device 100 to perform all or part of one or more methods and/or operations described herein. Said Vapor device is capable of measuring exact quantities of said liquid inhaled by said user and delivering exact “metered” dosages 120 of said liquid to said user.

In an embodiment, the vapor device 100 can comprise a network access device 106 allowing the vapor device 100 to be coupled to one or more secondary devices such as via an access point of a wireless telephone network, local area network, or other coupling to a wide area network, for example, the Internet. In that regard, the processor 102 can be configured to share data with the one or more secondary devices via the network access device 106. The shared data may comprise, for example, usage data and/or operational data of the vapor device 100, a status of the vapor device 100, a status and/or operating condition of one or more the components of the vapor device 100, text to be used in a message, a product order, payment information, and/or any other data. Similarly, the processor 102 can be configured to receive control instructions from the one or more secondary devices via the network access device 106. For example, a configuration of the vapor device 100, an operation of the vapor device 100, and/or other settings of the vapor device 100, can be controlled by the one or more secondary devices via the network access device 106. For example, a secondary device may comprise a server that can provide various services and another secondary device can comprise a smartphone or smart watch for controlling operation of the vapor device 100. In some embodiments, the smartphone or other secondary device can be used as a primary input/output of the vapor device 100 such that data is received by the vapor device 100 from the server, transmitted to the smartphone, and output on a display of the smartphone. In an embodiment, data transmitted from the secondary device 130 may comprise additional data and commands to said vaporizer 108 which determine when a metered amount 120 of said liquid may be inhaled by a user 126 through an outlet 144, such as a mouthpiece 60.

In an embodiment, the vapor device 100 may also comprise an input/output device 112 which may be the input 34 or output 32 devices described in FIG. 1 coupled to one or more of the processors 102, a vibratable mesh 50, the network access device 106, and/or any other electronic component of the vapor device 100. Input can be received from a user or another device and/or output can be provided to a user or another device via the input/output device 112. The input/output device 112 may comprise any combinations of input and/or output devices such as buttons, knobs, keyboards, touchscreens, displays, light-emitting elements, a speaker, and/or the like. In an embodiment, the input/output device 112 can comprise an interface port (not shown) such as a wired interface, for example a serial port, a Universal Serial Bus (USB) port, an Ethernet port, or other suitable wired connection. The input/output device 112 can comprise a wireless interface (not shown), for example a transceiver using any suitable wireless protocol, for example WIFI (IEEE 802.11), Bluetooth®, infrared, or other wireless standard. For example, the input/output device 112 can communicate with a smartphone via Bluetooth® such that the inputs and outputs of the smartphone can be used by the user to interface with the vapor device 100. In an embodiment, the input/output device 112 can comprise a user interface.

In an embodiment, the input/output device 112 can be coupled to an adaptor device to receive power and/or send/receive data signals from an electronic device. For example, the input/output device 112 can be configured to receive power from the adaptor device and provide the power to the power supply 120 to recharge one or more batteries. The input/output device 112 can exchange data signals received from the adaptor device with the processor 102 to cause the processor to execute one or more functions.

In an embodiment, the input/output device 112 can comprise a touchscreen interface and/or a biometric interface. For example, the input/output device 112 can include controls that allow the user to interact with and input information and commands to the vapor device 100. For example, with respect to the embodiments described herein, the input/output device 112 can comprise a touch screen display. The input/output device H2 can be configured to provide the content of the exemplary screen shots shown herein, which are presented to the user via the functionality of a display. User inputs to the touch screen display are processed by, for example, the input/output device 112 and/or the processor 102. The input/output device 112 can also be configured to process new content and communications to the system 100. The touch screen display can provide controls and menu selections, and process commands and requests. Application and content objects can be provided by the touch screen display. The input/output device 112 and/or the processor 102 can receive and interpret commands and other inputs, interface with the other components of the vapor device 100 as required. In an embodiment, the touch screen display can enable a user to lock, unlock, or partially unlock or lock, the vapor device 100. The vapor device 100 can be transitioned from an idle and locked state into an open state by, for example, moving or dragging an icon on the screen of the vapor device 100, entering in a password/passcode, and the like. The input/output device 112 can thus display information to a user such as a puff count, an amount of vaporizable material remaining in a container 110, battery remaining, signal strength, combinations thereof: and the like.

In an embodiment, the input/output device 112 can comprise an audio user interface. A microphone can be configured to receive audio signals and relay the audio signals to the input/output device 112. The audio user interface can be any interface that is responsive to voice or other audio commands. The audio user interface can be configured to cause an action, activate a function, etc, by the vapor device 100 (or another device) based on a received voice (or other audio) command. The audio user interface can be deployed directly on the vapor device 100 and/or via other electronic devices (e.g., electronic communication devices such as a smartphone, a smart watch, a tablet, a laptop, a dedicated audio user interface device, and the like). The audio user interface can be used to control the functionality of the vapor device 100. The audio user interface can also be utilized to create and send a custom message to other users, to join e-Vapor clubs, to receive e-Vapor chart information, and to conduct a wide range of social networking, location services and eCommerce activities. The audio user interface can be secured via a password (e.g., audio password) which features at least one of tone recognition, other voice quality recognition and, in one embodiment, can utilize at least one special cadence as part of the audio password.

The input/output device 112 can be configured to interface with other devices, for example, exercise equipment, computing equipment, communications devices and/or other vapor devices, for example, via a physical or wireless connection. The input/output device 112 can thus exchange data with the other equipment. A user may sync their vapor device 100 to other devices, via programming attributes such as mutual dynamic link library (DLL) ‘hooks’. This enables a smooth exchange of data between devices, as can a web interface between devices. The input/output device 112 can be used to upload one or more profiles to the other devices. Using exercise equipment as an example, the one or more profiles can comprise data such as workout routine data (e.g., timing, distance, settings, heart rate, etc. . . . ) and vaping data (e.g., e-Liquid mixture recipes, supplements, vaping timing, etc. . . . ). Data from usage of previous exercise sessions can be archived and shared with new electronic vapor devices and/or new exercise equipment so that history and preferences may remain continuous and provide for simplified device settings, default settings, and recommended settings based upon the synthesis of current and archival data.

In an embodiment, the vapor device 100 can comprise a global positioning system (GPS) unit 118. The GPS 118 can detect a current location of the device 100. In some embodiments, a user can request access to one or more services that rely on a current location of the user. For example, the processor 102 can receive location data from the GPS 118, convert it to usable data, and transmit the usable data to the one or more services via the network access device 106.

In an embodiment, the vapor device may comprise one or more sensors 136, sensors may include, a touch screen, a fingerprint identifier, button, a microphone, a heart rate monitor, a thermometer, or other sensors.

In an embodiment, the vapor device system may further comprise anonymizing data transmitted and received for HIPPA compliance and user privacy safety.

In an additional embodiment, a vapor device system may receive data from a second device generated by one or more users to predict a user's physical response to an administered material.

In one embodiment of the disclosure, a system can be configured to provide services such as network-related services to a user device. FIG. 3 illustrates various embodiments of an exemplary environment in which the present methods and systems can operate. The present disclosure is relevant to systems and methods for providing services to a user device, for example, electronic vapor devices which can include, but are not limited to, a vape-bot, micro-vapor device, vapor pipe, e-cigarette, hybrid handset and vapor device, and the like. Other user devices that can be used in the systems and methods include, but are not limited to, a smart watch (and any other form of “smart” wearable technology), a smartphone, a tablet, a laptop, a desktop, and the like. In an embodiment, one or more network devices can be configured to provide various services to one or more devices, such as devices located at or near a premises. In another embodiment, the network devices can be configured to recognize an authoritative device for the premises and/or a particular service or services available at the premises. As an example, an authoritative device can be configured to govern or enable connectivity to a network such as the Internet or other remote resources, provide address and/or configuration services like DHCP, and/or provide naming or service discovery services for a premises, or a combination thereof. Those skilled in the art will appreciate that present methods can be used in various types of networks and systems that employ both digital and analog equipment. One skilled in the art will appreciate that provided herein is a functional description and that the respective functions can be performed by software, hardware, or a combination of software and hardware.

The network and system can comprise a user device 1202a, 1202b, and/or 1202c in communication with a computing device 1204 such as a server, for example. The computing device 1204 can be disposed locally or remotely relative to the user device 1202a, 1202b, and/or 1202c. As an example, the user device 1202a, 1202b, and/or 1202c and the computing device 1204 can be in communication via a private and/or public network 1220 such as the Internet or a local area network. Other forms of communications can be used such as wired and wireless telecommunication channels, for example. In another embodiment, the user device 1202a, 1202b, and/or J 202c can communicate directly without the use of the network 1220 (for example, via Bluetooth®, infrared, and the like).

In an embodiment, the user device 1202a, 1202b, and/or 1202c can be an electronic device such as an electronic vapor device (e.g., vape-bot, micro-vapor device, vapor pipe, e-cigarette, hybrid handset and vapor device), a smartphone, a smart watch, a computer, a smartphone, a laptop, a tablet, a set top box, a display device, or other device capable of communicating with the computing device 1204. As an example, the user device 1202a, 1202b, and/or 1202c can comprise a communication element 1206 for providing an interface to a user to interact with the user device 1202a, 1202b, and/or 1202c and/or the computing device 1204. The communication element 1206 can be any interface for presenting and/or receiving information to/from the user, such as user feedback. An example interface can be a communication interface such as a web browser (e.g., Internet Explorer, Mozilla Firefox, Google Chrome, Safari, or the like). Other software, hardware, and/or interfaces can be used to provide communication between the user and one or more of the user devices 1202a, 1202b, and/or 1202c and the computing device 1204. In an embodiment, the user device 1202a, 1202b, and/or 1202c can have at least one similar interface quality such as a symbol, a voice activation protocol, a graphical coherence, a startup sequence continuity element of sound, light, vibration, or symbol. In an embodiment, the interface can comprise at least one of lighted signal lights, haptic feedback, sounds or other interface system functions.

As an example, the communication element 1206 can request or query various files from a local source and/or a remote source. As a further example, the communication element 1206 can transmit data to a local or remote device such as a computing device 1204.

In an embodiment, the user device 1202a, 1202b, and/or 1202c can be associated with a user identifier or device identifier 1208a, 1208b, and/or 1208c. As an example, the device identifier 1208a, 1208b, and/or 1208c can be any identifier, token, character, string, or the like, for differentiating one user or user device (e.g., user device 1202a, 1202b, and/or 1202c) from another user or user device. In a further embodiment, the device identifier 1208a, 1208b, and/or 1208c can identify a user or user device as belonging to a particular class of users or user devices. As a further example, the device identifier 1208a, 1208b, and/or 1208c can comprise information relating to the user device such as a manufacturer, a model or type of device, a service provider associated with the user device 1202a, 1202h, and/or 1202c, a state of the user device 1202a, 1202b, and/or 1202c, a locator, and/or a label or classifier. Other information can be represented by the device identifier 1208a, 1208b, and/or 1208c.

In an embodiment, the device identifier 1208a, 1208b, and/or 1208c can comprise an address element 1210 and a service element 1212. In an embodiment, the address element 1210 can comprise or provide an internet protocol address, a network address, a media access control (MAC) address, an Internet address, or the like. As an example, the address element 1210 can be relied upon to establish a communication session between the user device 1202a, 1202b, and/or 1202c and the computing device 1204 or other devices and/or networks. As a further example, the address element 1210 can be used as an identifier or locator of the user device 1202a, 1202b, and/or 1202c. In an embodiment, the address element 1210 can be persistent for a particular network.

In an embodiment, the service element 1212 can comprise an identification of a service provider associated with the user device 1202a, 1202b, and/or 1202c and/or with the class of user device 1202a, 1202b, and/or 1202. The class of the user device 1202a, 1202b, and/or 1202c can be related to a type of device, capability of device, type of service being provided, and/or a level of service. As an example, the service element 1212 can comprise information relating to or provided by a communication service provider (e.g., Internet service provider) that is providing or enabling data flow such as communication services to and/or between the user device 1202a, 1202b, and/or 1202c. As a further example, the service element 1212 can comprise information relating to a preferred service provider for one or more particular services relating to the user device 1202a, 1202b, and/or 1202c. In an embodiment, the address element 1210 can be used to identify or retrieve data from the service element 1212, or vice versa. As a further example, one or more of the address elements 1210 and the service element 1212 can be stored remotely from the user device 1202a, 1202b, and/or 1202c and retrieved by one or more devices such as the user device 1202a, 1202b, and/or 1202c and the computing device 1204. Other information can be represented by the service element 1212.

In an embodiment, the computing device 1204 can be a server for communicating with the user device 1202a, 1202b, and/or 1202c. As an example, the computing device 1204 can communicate with the user device 1202a, 1202b, and/or 1202c for providing data and/or services. In an embodiment, the computing device 1204 can allow the user device 1202a, 1202b, and/or 1202c to interact with remote resources such as data, devices, and files. As an example, the computing device can be configured as (or disposed at) a central location, which can receive content (e.g., data) from multiple sources, for example, user devices 1202a, 1202b, and/or 1202c. The computing device 1204 can combine the content from the multiple sources and can distribute the content to user (e.g., subscriber) locations via a distribution system.

In an embodiment, one or more network devices 1216 can be in communication with a network such as network 1220. As an example, one or more of the network devices 1216 can facilitate the connection of a device, such as user device 1202a, 1202b, and/or 1202c, to the network 1220. As a further example, one or more of the network devices 1216 can be configured as a wireless access point (WAP). In an embodiment, one or more network devices 1216 can be configured to allow one or more wireless devices to connect to a wired and/or wireless network using Wi-Fi, Bluetooth, or any desired method or standard.

In an embodiment, the network devices 1216 can be configured as a local area network (LAN). As an example, one or more network devices 1216 can comp lise a dual band wireless access point. As an example, the network devices 1216 can be configured with a first service set identifier (SSID) (e.g., associated with a user network or private network) to function as a local network for a particular user or users. As a further example, the network devices 1216 can be configured with a second service set identifier (SSID) (e.g., associated with a public/community network or a hidden network) to function as a secondary network or redundant network for connected communication devices.

In an embodiment, one or more network devices 1216 can comprise an identifier 1218. As an example, one or more identifiers can be or relate to an Internet Protocol (IP) Address IPV4/IPV6 or a media access control address (MAC address) or the like. As a further example, one or more identifiers 1218 can be a unique identifier for facilitating communications on the physical network segment. In an embodiment, each of the network devices 1216 can comprise a distinct identifier 1218. As an example, the identifiers 1218 can be associated with a physical location of the network devices 1216.

In an embodiment, the computing device 1204 can manage the communication between the user device 1202a, 1202b, and/or 1202c and a database 1214 for sending and receiving data therebetween. As an example, the database 1214 can store a plurality of files (e.g., web pages), user identifiers or records, or other information. In one embodiment, the database 1214 can store user device 1202a, 1202b, and/or 1202c usage information (including chronological usage), type of vaporizable and/or non-vaporizable material used, frequency of usage, location of usage, recommendations, communications (e.g., text messages, advertisements, photo messages), simultaneous use of multiple devices, and the like); as well as weight data, body fat data, heart rate data, blood pressure data, temperature data, movement data, sleep data, blood sugar data, and medication data; and combinations thereof which may be transmitted to a vapor device and used to determine when a metered amount of said liquid may be inhaled by a user.

The database 1214 can collect and store data to support cohesive use, wherein cohesive use is indicative of the use of a first electronic vapor devices and then a second electronic vapor device is synced chronologically and logically to provide the proper specific properties and amount of vapor based upon a designed usage cycle. As a further example, the user device 1202a, 1202b, and/or 1202c can request and/or retrieve a file from the database 1214. The user device 1202a, 1202b, and/or 1202c can thus sync locally stored data with more current data available from the database 1214. Such syncing can be set to occur automatically on a set time schedule, on demand, and/or in real-time. The computing device 1204 can be configured to control syncing functionality. For example, a user can select one or more of the user devices 1202a, 1202b, and/or 1202c to never by synced, to be the master data source for syncing, and the like. Such functionality can be configured to be controlled by a master user and any other user authorized by the master user or agreement.

In an embodiment, data can be derived by system and/or device analysis. Such analysis can comprise at least by one of instant analysis performed by the user device 1202a, 1202b, and/or 1202c or archival data transmitted to a third party for analysis and returned to the user device 1202a, 1202b, and/or 1202c and/or computing device 1204. The result of either data analysis can be communicated to a user of the user device 1202a, 1202b, and/or 1202c to, for example, inform the user of their e-Vapor use and/or lifestyle options. In an embodiment, a result can be transmitted back to at least one authorized user interface.

In an embodiment, the database 1214 can store information relating to the user device 1202a, 1202b, and/or 1202c such as the address element 1210 and/or the service element 1212. As an example, the computing device 1204 can obtain the device identifier 1208a, 1208b, and/or 1208c from the user device 1202a, 1202b, and/or 1202c and retrieve information from the database 1214 such as the address element 1210 and/or the service elements 1212. As a further example, the computing device 1204 can obtain the address element 1210 from the user device 1202a, 1202b, and/or 1202c and can retrieve the service element 1212 from the database 1214, or vice versa. Any information can be stored in and retrieved from the database 1214. The database 1214 can be disposed remotely from the computing device 1204 and accessed via direct or indirect connection. The database 1214 can be integrated with the computing device 1204 or some other device or system.

FIG. 4 illustrates an ecosystem 1300 configured for sharing and/or syncing data such as usage information (including chronological usage), type of vaporizable and/or non-vaporizable material used, frequency of usage, location of usage, recommendations, communications (e.g., text messages, advertisements, photo messages), simultaneous use of multiple devices, and the like) between one or more devices such as a vapor device 1302, a vapor device 1304, a vapor device 1306, and an electronic communication device 1308; additionally weight data, body fat data, heart rate data, blood pressure data, temperature data, movement data, sleep data, blood sugar data, and medication data; and combinations thereof may be shared and synced allowing for determination of when a metered amount of said liquid may be inhaled by a user.

In an embodiment, the vapor device 1302, the vapor device 1304, the vapor device 1306 can be one or more of an e-cigarette, an e-cigar, an electronic vapor modified device, a hybrid electronic communication handset coupled/integrated vapor device, a micro-sized electronic vapor device, or a robotic vapor device. In an embodiment, the electronic communication device 1308 can comprise one or more of a smartphone, a smart watch, a tablet, a laptop, and the like.

In an embodiment data generated, gathered, created, etc, by one or more of the vapor devices 1302, the vapor device 1304, the vapor device 1306, and/or the electronic communication device 1308 can be uploaded to and/or downloaded from a central server 1310 via a network 1312, such as the Internet. Such uploading and/or downloading can be performed via any form of communication including wired and/or wireless. In an embodiment, the vapor device 1302, the vapor device 1304, the vapor device 1306, and/or the electronic communication device 1308 can be configured to communicate via cellular communication, WIFI communication, Bluetooth communication, satellite communication, and the like. The central server 1310 can store uploaded data and associate the uploaded data with a user and/or device that uploaded the data. The central server 1310 can access unified account and tracking information to determine devices that are associated with each other, for example devices that are owned/used by the same user. The central server 1310 can utilize the unified account and tracking information to determine which of the vapor device 1302, the vapor device 1304, the vapor device 1306, and/or the electronic communication device 1308, if any, should receive data uploaded to the central server 1310.

For example, the vapor device 1302 can be configured to upload usage information related to vaporizable material consumed and the electronic communication device 1308 can be configured to upload location information related to location of the vapor device 1302. The central server 1310 can receive both the usage information and the location information, access the unified account and tracking information to determine that both the vapor device 1302 and the electronic communication device 1308 are associated with the same user. The central server 1310 can thus correlate the user's location along with the type, amount, and/or timing of usage of the vaporizable material. The central server 1310 can further determine which of the other devices are permitted to receive such information and transmit the information based on the determined permissions. In an embodiment, the central server 1310 can transmit the correlated information to the electronic communication device 1308 which can then subsequently use the correlated information to recommend a specific type of vaporizable material to the user when the user is located in the same geographic position indicated by the location information.

In another embodiment, the central server 1310 can provide one or more social networking services for users of the vapor device 1302, the vapor device 1304, the vapor device 1306, and/or the electronic communication device 1308. Such social networking services include, but are not limited to, messaging, text, images, and/or video, location sharing, product ordering, and the like.

As illustrated in FIG. 5, the apparatus or system 1600 can comprise an electrical component 1602 for determining data from the sensor of a vaporizer apparatus. The component 1602 may be, or may include a feature for detecting data, interpreting data and/or transmitting data. Said feature may include a sensor 1618, the processor 1610 coupled to the memory 1616, and the network interface 1614, the processor executing an algorithm based on program instructions stored in the memory. Such algorithm may include a sequence of more detailed operations, for example, the methods described herein.

The apparatus 1600 may optionally include a processor module 1610 having at least one processor, in the case of the apparatus 1600 configured as a controller for a fluid dispenser 1618. The processor 1610, in such case, may be in operative communication with the memory 1616, interface 1614 or the sensor 1618 via a bus 1612 or similar communication coupling. The processor 1610 may affect initiation and scheduling of the processes or functions performed by the electrical component 1602.

In related embodiments, the apparatus 1600 may include a network interface module operable for communicating with a server over a computer network. The apparatus may include a controllable dispenser for a vaporizable material, for example, a nebulizer for which vaporization rate is correlated to power supplied, or a micro-valve for which vaporization is proportional to valve position. In further related embodiments, the Vapor device 1600 may optionally include a module for storing information, such as, for example, a memory device/module 1616. The computer readable medium or the memory module 1616 may be operatively coupled to the other components of the Vapor device 1600 via the bus 1612 or the like. The memory module 1616 may be adapted to store computer readable instructions and data for enabling the processes and behavior of the module 1602, and subcomponents thereof, or the processor 1610, or the methods described herein. The memory module 1616 may retain instructions for executing functions associated with the module 1602. While shown as being external to the memory 1616, it is to be understood that the module 1602 can exist within the memory 1616.

In view of the exemplary systems described supra, methodologies that can be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks can be required to implement the methodologies described herein. Additionally, it should be further appreciated that the methodologies disclosed herein are capable of being stored on an article of manufacture to facilitate transporting and transferring such methodologies to computers.

Those of skill would further appreciate that the various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

As used in this application, the terms “component,” “module,” “system,” and the like are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components may reside within a process and/or thread of execution and a component can be localized on one computer and/or distributed between two or more computers.

As used herein, a “vapor” includes mixtures of a carrier gas or gaseous mixture (for example, air) with any one or more of a dissolved gas, suspended solid particles, or suspended liquid droplets, wherein a substantial fraction of the particles or droplets if present are characterized by an average diameter of not greater than three microns. As used herein, an “aerosol” has the same meaning as “vapor,” except for requiring the presence of at least one of particles or droplets. A substantial fraction means 10% or greater; however, it should be appreciated that higher fractions of small (<3 micron) particles or droplets can be desirable, up to and including 100—It should further be appreciated that, to simulate smoke, average particle or droplet size can be less than three microns, for example, can be less than one micron with particles or droplets distributed in the range of 0.01 to 1 micron. A vaporizer may include any device or assembly that produces a vapor or aerosol from a carrier gas or gaseous mixture and at least one vaporizable material.

Various embodiments presented in terms of systems can comprise a number of components, modules, and the like. It is to be understood and appreciated that the various systems may include additional components, modules, etc. and/or may not include all of the components, modules, etc. discussed in connection with the figures. A combination of these approaches can also be used.

In addition, the various illustrative logical blocks, modules, and circuits described in connection with certain embodiments disclosed herein can be implemented or performed with a general purpose processor, a digital signal processor (DSP), an application specific integrated circuit (ASIC), a field programmable gate army (FPGA) or other programmable logic device, discrete gate or transistor logic, discrete hardware components, or any combination thereof designed to perform the functions described herein. A general-purpose processor can be a microprocessor, but in the alternative, the processor can be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

The scope of the present disclosure fully encompasses other embodiments which may become obvious to those skilled in the art, and is to be limited, accordingly, by nothing other than the appended claims. All structural and functional equivalents to the elements of the above-described preferred embodiment and additional embodiments as regarded by those of ordinary skill in the art are hereby expressly incorporated by reference and are intended to be encompassed by the present claims. Furthermore, no element, component, or method step in the present disclosure is intended to be dedicated to the public regardless of whether the element, component, or method step is explicitly recited in the claims.

What is claimed is:

1. A vapor device system, comprising:
a cartridge section comprising: a reservoir containing a liquid to be vaporized and a mouthpiece whereby a user may inhale vaporized liquid from the mouthpiece;
a vibratable mesh, said vibratable mesh being disposable between said reservoir and said mouthpiece, so as to vaporize liquid drawn from the reservoir to the mouthpiece;
a flow sensor, wherein said flow sensor is arranged to detect a user drawing fluid through the mouthpiece during use and to actuate the vibrating mesh when fluid flow is detected;
a body section releasably connectable to said cartridge section, further comprising:
a processor;
a memory device;
a network access device;
an input and output device;
a power supply;
wherein said vapor device system records data on the amount of said liquid inhaled by said user and transmits said data to a second device and wherein said second device transmits additional data back to said vapor device system which determine when a metered amount of said liquid may be inhaled by said user.

2. The vapor device system of claim 1, wherein said vapor device system records data on the identity of said user and transmits said data to a second device and wherein said second device transmits additional data back to said vapor device system which determine when a metered amount of said liquid may be inhaled by said user.

3. The vapor device system of claim 2, wherein said second device transmits data to said vapor device system selected from the group consisting of: weight data, body fat data, heart rate data, blood pressure data, temperature data, movement data, sleep data, blood sugar data, and medication data; and combinations thereof which determine when a metered amount of said liquid may be inhaled by said user.

4. The vapor device system of claim 3, further comprising receiving data from a second device generated by one or more users to predict a user's physical response to an administered material.

5. The vapor device system of claim 2, further comprise a step of anonymizing data transmitted.

6. The vapor device system of claim 3, further comprise a step of anonymizing data received.

7. The vapor device system of claim 1, wherein said vapor device system records data on the content of said liquid and transmits said data to a second device and wherein said second device transmits additional data back to said vapor device system which determine when a metered amount of said liquid may be inhaled by said user.

8. The vapor device system of claim 7, wherein said cartridge contains an indication of the contents of the liquid therein and transmits that to said vapor device system.

9. The vapor device system of claim 7, wherein said second device transmits data to said vapor device system selected from the group consisting of: weight data, body fat data, heart rate data, blood pressure data, temperature data, movement data, sleep data, blood sugar data, and medication data; and combinations thereof which determine when a metered amount of said liquid may be inhaled by said user.

10. The vapor device system of claim 9, further comprising receiving data from a second device generated by one or more users to predict a user's physical response to an administered material.

11. The vapor device system of claim 7, wherein said vapor device system records data on the identity of said user and transmits said data to a second device and wherein said second device transmits additional data back to said vapor device system selected from the group consisting of: weight data, body fat data, heart rate data, blood pressure data, temperature data, movement data, sleep data, blood sugar data, and medication data; and combinations thereof which determine when a metered amount of said liquid may be inhaled by said user.

12. The vapor device system of claim 11, wherein said cartridge contains an indication of the contents of the liquid therein and transmits that to said vapor device system.

13. The vapor device system of claim 12, further comprising receiving data from a second device generated by one or more users to predict a user's physical response to an administered material.

14. The vapor device system of claim 1, wherein said output device informs the user when and how much of said metered amount of liquid is to be used.

15. The vapor device system of claim 14, wherein said output device is a light.

16. The vapor device system of claim 14, wherein said output device is a sound.

17. The vapor device system of claim 14, wherein said output device is haptic feedback.

18. The vapor device system of claim 1, wherein said liquid is water based.

19. The vapor device system of claim 1, wherein said second device is a smart phone.

20. The vapor device system of claim 1, wherein said second device is a smart watch.
